(19) United States
(12) Patent Application Publication
(10) Pub. No.: US 2009/0263891 A1
(43) Pub. Date: Oct. 22, 2009

(54) DEVICE FOR ANALYZING SAMPLES INCLUDING A GAS EVOLVING MEANS

(75) Inventors: Murray Fulton Gillies, Eindhoven (NL); Erik Robbert Vossenaar, De Meern (NL); Mark Thomas Johnson, Veldhoven (NL); Hendrik Roelof Stapert, Eindhoven (NL); David Andrew Fish, Haywards Heath (GB); Ralph Kurt, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/373,993

(22) PCT Filed: Mar. 12, 2007
(86) PCT No.: PCT/IB2007/050808

§ 371 Date: Jan. 15, 2009

(30) Foreign Application Priority Data
Jul. 19, 2006 (EP) 06117464.5

(51) Int. Cl. C12M 1/34

(52) U.S. Cl. 435/287.2

(57) ABSTRACT

The invention relates to a device for analyzing one or more samples for the presence, amount or identity of one or more target molecules in the samples, comprising one or more capture sites whereby the device comprises a gas evolving means. The gas evolved by the gas evolving means moves unbound target molecules away from the capture site and therefore helps to increase the efficacy of the analysis.

### FIELD OF THE INVENTION

The present invention is directed to the field of devices for the detection of one or more target molecules in a fluid sample, especially to the field of devices for the detection of biomolecules in aqueous solution. Furthermore the present invention is directed to a control circuit to simplify the analysis of the sample.

### BACKGROUND OF THE INVENTION

The present invention is directed to the detection of target molecules in fluids, especially to the detection of biomolecules in aqueous solution. The detection usually occurs in such a way that the fluid to be analyzed is provided on a substrate material, which contains capture sites for the target molecules which are subject of the detection. Such a capture site may be a corresponding DNA-strand in case the target molecule is also a DNA-Strand or an antibody in the case of a protein assay. The target molecules in the fluid will then bind to the capture site and remain there even after the fluid is removed. The target molecule contains a label and in this way may be detected.

However, especially if small volumes of samples are to be detected, the removal of non-bound but marked species in the sample is a problem, since these compounds must be removed in order not to cause a later misreading. In prior art, therefore excessive washing steps are common which on the one hand lengthen the analysis procedure and on the other hand add complexity and cost to the analytical device.

### SUMMARY OF THE INVENTION

It is therefore an object of the present invention to provide a device, which allows a quicker detection with a minute amount of target molecule that needs to be present in the fluid.

This object is solved by a device according to claim 1 of the present invention. Accordingly, a device for analyzing one or more samples, especially fluid samples for the presence, amount or identity of one or more target molecules in the samples is provided, comprising one or more capture sites whereby the device comprises a gas evolving means for washing away and/or removing unbound target molecules and/or redundant sample fluid from the capture site.

Surprisingly it has been shown that it is for most applications within the present invention possible to remove non-bound species from the capture sites by guiding gas over them whilst bound target molecules will not be removed.

A substrate material according to the present invention has the following advantages over the prior art:
The removal of non-bound species is efficient and time-effective, thus reducing the amount of time needed for the analysis
No washing agent or associated microfluidics/reservoir are required.
The outcoupling of fluorescent light is improved as no light is guided away from the detector through the sample liquid.

As will be appreciated by those in the art, the sample may comprise any number of things, including, but not limited to, bodily fluids (e.g. blood, urine, serum, lymph, saliva, anal and vaginal secretions, perspiration and semen) of virtually any organism, with mammalian samples being preferred and human samples particularly preferred; environmental samples (e.g. air, agricultural, water and soil samples); biological warfare agent samples; research samples (i.e. in the case of nucleic acids).

As will be appreciated by those in the art, the target molecule(s) may be, but not limited to, the product(s) of an amplification reaction, including both target and signal amplification); purified samples, such as purified genomic DNA, RNA, proteins, etc.; raw samples (bacteria, virus, genomic DNA, etc.); biological molecular compounds such as, but not limited to, nucleic acids and related compounds (e.g. DNAs, RNAs, oligonucleotides or analogs thereof, PCR products, genomic DNA, bacterial artificial chromosomes, plasmids and the like), proteins and related compounds (e.g. polypeptides, peptides, monoclonal or polyclonal antibodies, soluble or bound receptors, transcription factors, and the like), antigens, ligands, haptens, carbohydrates and related compounds (e.g. polysaccharides, oligosaccharides and the like), cellular fragments such as membrane fragments, cellular organelles, intact cells, bacteria, viruses, protozoa, and the like.

As will be appreciated by those in the art, virtually any experimental manipulation may have been done on the sample and/or the target molecules.

According to a preferred embodiment of the present invention, the gas evolved by the gas evolving means is created by electrolysis of the sample solution.

According to a preferred embodiment of the present invention, the gas evolving means evolves gas by applying a voltage of ≧1.2 V and ≦10V. This has been shown to be a suitable voltage range in order to obtain suitable gas bubbles in a reproducible manner. According to a preferred embodiment of the present invention, the gas evolving means evolves gas by applying a voltage of ≧2 V and ≦4.5V, according to a preferred embodiment of the present invention, the gas evolving means evolves gas by applying a voltage of ≧2.5 V and ≦3.5V.

According to a preferred embodiment of the present invention, the gas evolving means evolves gas by using a current of ≧10⁻⁸ A and ≦10⁻⁶ A.

This has been shown within a wide range of applications to be a suitable voltage range in order to obtain suitable gas bubbles in a reproducible manner.

According to a preferred embodiment of the present invention, the gas evolving means evolves gas by using a current of ≧2.5*10⁻⁸ A and ≦7.5*10⁻⁷ A, According to a preferred embodiment of the present invention, the gas evolving means evolves gas by using a current of ≧5*10⁻⁸ A and ≦5*10⁻⁷ A.

According to a preferred embodiment of the present invention, the gas evolving part of the gas evolving means is located in a distance of ≧0 mm and ≦5 mm from the capture sites. This distance has been shown to be advantageous in practice for a wide range of applications within the present invention. According to a preferred embodiment of the present invention, the gas evolving part of the gas evolving means is located in a distance of ≧10 μm and ≦1 mm, preferably ≧100 μm and ≦500 μm from the capture sites.

It should be noted that in case the gas evolvement is achieved via electrolysis of the sample solution, which will be in most applications water based, according to a preferred embodiment of the present solution, only the hydrogen evolving part of the gas evolving means will be used whereas the oxygen evolving part is preferably located more remote from the capture sites or according to another preferred embodiment located in a separate component of the device.

In embodiments within the present invention which use a gas evolving means of this composition, it is preferred that the hydrogen evolving part of the gas evolving means is located in a distance of ≧0 mm and ≦5 mm from the capture sites. This distance has been shown to be advantageous in practice for a wide range of applications within the present invention. According to a preferred embodiment of the present invention, the gas evolving part of the gas evolving means is located in a distance of ≧10 μm and ≦1 mm, preferably ≧100 μm and ≦500 μm from the capture sites.

In some applications within the present invention, oxygen may be harmful either to the capture sites, the target molecules or the labels, which are in most application fluorescent dyes some of which are known to be quite sensitive to oxygen. Therefore it is preferred if only the hydrogen is used within the gas evolving means whereas the oxygen is held in solution and/or evolved at a remote part of the device.

According to a preferred embodiment of the present invention, the gas evolving means comprises at least one pair of electrodes.

According to a preferred embodiment of the present invention, the gas evolving means is also adapted to serve as a means to transport molecules, especially the target molecules to the capture site. The method for transportation may according to one embodiment of the present invention be provided in the form of electrophoretic transport (if the particles are charged as is the case for DNA and proteins) or via di-electrophoresis for uncharged molecules. Such uncharged molecules can for example be beads coated with antibodies. In the latter case it is preferred that a traveling wave with four phases is used Alternatively pressure differences created by the local generation of gas can according to one embodiment of the present invention be used to move the solution containing the target molecules and so create mixing and improve the chance of capturing.

Alternatively or additionally and insofar another embodiment of the present invention electro-osmotic movement may be generated to create mixing and improve the chance of capturing.

The present invention is also directed to a gas evolution control means in particular for removing unbound target molecules and/or redundant sample fluid from at least one capture site of a device according to the present invention including a feedback mechanism controlling the formation and/or the movement of evolved gas bubbles.

However, it should be noted that such a gas evolution control means is also of inventive use without such a device and may be employed in a wide range of other applications.

It is well known that the nucleation of gas bubbles from a liquid is within a wide range of applications not a well defined process and can be rather unpredictable as it depends on many device parameters such as the surface of the gas evolving means and/or whether there is significant local roughness or conductivity of the sample liquid. Furthermore, it is for a wide range of applications not possible or very difficult to allow one capture site to further hybridize, since the signal is weak, while stopping another side where the signal has reached its maximum.

Therefore it is according to one embodiment of the present invention advantageous to provide such a gas evolution control means to allow a controlled development of the gas bubbles.

In one preferred embodiment of the present invention the control means includes a feedback mechanism to accurately control the amount and/or the formation and/or the movement of the gas bubbles. The feedback mechanism may be for example at least one active control circuit and/or may comprise at least one activating means.

According to a preferred embodiment of the present invention the gas evolution control means comprises at least two electrodes for said gas evolution and the feedback mechanism comprises an impedance network of at least one capacitor and/or resistor which is/are located between said electrodes to measure a capacity and/or a resistance between said electrodes to control the formation and/or the movement of the gas bubble.

This feedback mechanism may control the amount and/or the movement of gas evolved by the gas evolving means by measuring the change for example in capacitance and/or resistance between two neighboring electrodes and comparing the value with a predetermined value.

The gas evolution control means in one embodiment of the present invention may be adapted in such a way that upon a change of the impedance, resistance and/or the capacity a signal is initiable by said activating means to activate a third electrode.

Since the sample solvent (which in most applications will be water based) has a greatly different resistance than the gas evolved by the gas evolving means, the difference in capacity and/or resistance can be measured easily and therefore it is possible to determine very accurately how far the “gas front” has progressed along the electrodes.

In a further embodiment of the present invention the electrodes are arranged as an active matrix such as a Large Area Electronic (LAE) technologies for example LTPS so that it is possible to individually address several electrodes at each capture side.

Advantageously in such a matrix the sample fluid at each side can be evacuated at any point in time. This allows either stopping of the hybridization at the maximum signal level or alternatively creating a sequence of otherwise identical sides but where the hybridization has been stopped at different points in time and so allowing the kinetics of the hybridization to be characterized.

According to a further embodiment of the present invention, the device further comprises at least one gas guiding means.

The term “gas guiding means” in the context of the present invention especially means and/or includes any means which is capable of guiding at least a part of the gas evolved from the gas evolving means at least partly along a preselected path on or with the device.

By doing so, it is for a wide range of applications within the present invention possible to build a more compact device and/or to increase the time of detection.

According to a further embodiment of the present invention, the gas guiding means is capable of selecting the size of the gas bubbles in that way that gas bubbles which have are certain size or larger are guided by the gas guiding means, whereas smaller gas bubbles remain essentially non-guided.

According to a further embodiment of the present invention, the gas guiding means is capable of guiding gas bubbles with an average size of ≧1 μm and ≦500 μm, preferably ≧5 μm and ≦100 μm and most preferred ≧10 μm and ≦50 μm, whereas smaller and/or larger gas bubbles remain essentially non-guided.

In this context the term “size” especially means the smallest dimension in case the bubbles are confirmed by the geometry and/or other features of the gas guiding means.

By doing so, it is for a wide range of applications within the present invention possible to have a faster and/or more efficient removal of unbound target molecules from the capture sites.

According to a further embodiment of the present invention, the gas evolving means evolves a plurality of gases and the gas guiding means is capable of selecting between the plurality of gases in that way that some gases are guided by the gas guiding means whereas others are not.

By doing so, it is in case that a plurality of gases are evolved by the gas evolving means for a wide range of applications possible to avoid disadvantages due to potentially reactive or otherwise harmful gases evolved by the gas evolving means since these gases will not be guided by the gas guiding means.

According to a further embodiment of the present invention, the gas evolving means evolves by hydrolysis of water and the gas guiding means is adapted to essentially guide only hydrogen whereas oxygen remains essentially unguided.

According to a further embodiment of the present invention, the gas evolving means comprises a hydrogel material.

The term “hydrogel material” in the sense of the present invention especially means and/or includes a network of polymer chains that are water-soluble.

According to an embodiment of the present invention, the hydrogel material comprises a poly(meth)acrylic material.

According to an embodiment of the present invention, the hydrogel material comprises a poly(meth)acrylic material made out of the polymerization of at least one (meth)acrylic monomer and at least one polyfunctional (meth)acrylic monomer.

According to an embodiment of the present invention, the (meth)acrylic monomer is chosen out of the group comprising (meth)acrylamide, (meth)acrylic acid, hydroxyethyl(meth)acrylate, ethoxyethoxyethyl(meth)acrylate or mixtures thereof.

According to an embodiment of the present invention, the polyfunctional (meth)acrylic monomer is a bis-(meth)acryl and/or a tri-(meth)acryl and/or a tetra-(meth)acryl and/or a penta-(meth)acryl monomer.

According to an embodiment of the present invention, the polyfunctional (meth)acrylic monomer is chosen out of the group comprising bis(meth)acrylamide, tripropyleneglycol di(meth)acrylates, pentaerythritol tri(meth)acrylate polyethyleneglycoldi(meth)acrylate, ethoxylated bisphenol-A-di(meth)acrylate or mixtures thereof.

According to an embodiment of the present invention, the crosslink density in the poly(meth)acrylic material is ≧0.05 and ≦1.

In the sense of the present invention, the term “crosslink density” means or includes especially the following definition: The crosslink density δ_{X} is here defined as

$${δ}_{X} = \frac{X}{L + X}$$

where X is the mole fraction of polyfunctional monomers and L the mole fraction of linear chain (=non polyfunctional) forming monomers. In a linear polymer δ_{X}=0, in a fully crosslinked system δ_{X}=1.

According to a further embodiment of the present invention, the gas evolving means comprises a nanoporous material.

According to a preferred embodiment of the present invention, the nanoporous material comprises an elastic polymeric material, preferably selected out of the group comprising rubbers, elastomers, a polymeric resist material, a polyacrylic material or mixtures of these materials.

According to an embodiment of the present invention, the hydrogel and/or nanoporous material has an open porosity of ≧0% and ≦70%. This has for a wide range of applications furthermore increased the suitability of the hydrogel material and/or nanoporous material for the gas guiding means.

According to an embodiment of the present invention, the hydrogel material and/or nanoporous material has a porosity of ≧1% and ≦40%, more preferred ≧3% and ≦20%.

In the sense of the present invention, the term “porosity” especially means or includes the ratio of the volume of all the pores or voids in a material to the volume of the whole. In other words, porosity is the proportion of the non-solid volume to the total volume of material. In the sense of the present invention porosity is especially a fraction between 0% and 100%.

According to a preferred embodiment of the present invention, the average pore size of the pores inside the hydrogel and/or nanoporous material is ≧10 nm and ≦2 μm, preferably ≧50 nm and ≦1 μm and most preferred between ≧100 nm and ≦500 nm

According to an embodiment of the present invention, the hydrogel and/or nanoporous material may comprise and/or be provided in an environment comprising water and small ions during operation.

According to an embodiment of the present invention, the hydrogel material and/or nanoporous material furthermore comprises an emulsion stabilizer. It has been shown that the addition of an emulsion stabilizer, especially during the build-up of the hydrogel material and/or nanoporous material helps to control the porosity for a wide range of applications within the present invention.

According to an embodiment of the present invention, the emulsion stabilizer is selected out of a group comprising polyether, alkylated polyether and mixtures thereof.

According to an embodiment of the present invention, hydrogel material and/or nanoporous material has an electrical conductivity in aqueous solution (i.e. in wet environment during operation) of ≧10 μS/m and ≦5 S/m, preferably ≧100 μS/m and ≦1 S/m, most preferably ≧1 mS/m and ≦500 mS/m.

According to an embodiment of the present invention, said electrical conductivity of the hydrogel material and/or nanoporous material is less than a factor 10, preferably less that a factor 5, different from the electrical conductivity of the analyte in aqueous solution (i.e. in wet environment during operation).

According to an embodiment of the present invention, the hydrogel material and/or nanoporous material is arranged as a layer on top of a substrate, the later comprising the electrodes. The layer thickness ranges preferably between ≧1 μm and ≦1 mm (in the wet state), more preferably between ≧10 μm and ≦500 μm, most preferably between ≧30 μm and ≦200 μm.

According to an embodiment of the present invention, the hydrogel or the alternative material for the gas guiding means is an elastic material with an elastic modulus E (in wet state)>100 kPa and <500 MPa, preferably E>1 MPa and <100 MPa.

It has been found that for most application the gas formation and guiding is increased in this way. Preferably when the channel is closed, when no gas is formed and open when the gas is formed.

According to an embodiment of the present invention, the gas guiding means comprises a connecting material, which helps to connect the hydrogel material with the device.

According to an embodiment of the present invention, the connecting material is not uniformly spread amongst the device, but the device involves regions, where connecting material is present and further regions, where no connecting material is present.

By doing so, the arrangement of the gas guiding means can for a wide range of applications be simplified very easily in that in that regions with the device which are along the preselected path, (and which are hereforth called “channel”) where gas is to be guided no connecting material is employed, whereas in the regions adjacent to these regions, the hydrogel material is connected to the device via the connecting material.

According to an embodiment of the present invention the channel(s) are arranged in a way that they form a dead end configuration, with the gas formation region in the end of the channel (and the capture probes along the channel or on outlet of the channel).

According to an embodiment of the present invention, the hydrogel material and/or nanoporous material is forming an elongated channel on top of a substrate, said channel has a width of ≧1 to ≦500 μm, preferably ≧50 to ≦100 μm.

According to an embodiment of the present invention, the device comprises a glass substrate and a plurality of electrodes located on said substrate and the connecting material is primarily provided on the part of the glass substrate, which are not covered by the plurality of electrodes.

According to an embodiment of the present invention, the connecting material comprises a silane material. By doing so it is for a wide range of applications greatly facilitated to apply the connecting material primarily on the part of the glass substrate which are not covered by the plurality of electrodes, since then the silane will react and/or bind to the glass surface, whereby no or essentially no reaction or binding with the electrodes will occur.

According to an embodiment of the present invention, the connecting material is selected out of a material chosen out of the group comprising methacrylicmethoxsilane, aminomethoxysilane, trimethoxysilyl-propyl-methacrylate or mixtures thereof. In this regard, the U.S. Pat. No. 6,960,298 is incorporated by reference.

According to an embodiment of the present invention said connecting material is pattern wise printed (e.g. by ink jet or microcontact printing or the like) onto said substrate prior to the deposition of said nanoporous or hydrogel layer forming the gas bubble guiding means. This pattering method is furthermore described in great detail in the WO 2005/015295 and literature cited therein, which is hereby fully incorporated by reference.

According to an embodiment of the present invention in the gas bubble formation region there are gas nucleation means provided for an improved gas nucleation out the liquid phase.

According to an embodiment of the present invention said gas nucleation means comprises local surface roughness or other topological surface modifications (such as small tips or needles) or small (50 nm-5 μm) particles such as dust, sand, glass or salt particles.

The present invention furthermore relates to a method for analyzing one or more samples especially fluid samples for the presence, amount or identity of one or more target molecules in the samples, comprising the steps of
(a) providing a device as described above
(b) adding an amount of sample to the substrate material, preferably to one or more defined areas on the substrate material
(c) allowing the target molecule in the samples to possibly combine with one or more of the capture sites
(d) evolving gas from the gas evolving means to remove non-bound target molecules, optionally using a gas guiding means
(e) optionally controlling and finally stopping the gas evolution via the gas evolution control means.

By doing so a quick and reliable detection of target molecules in the sample, even in minute amounts, is feasible for most applications within the present invention.

A substrate material, a method, a gas evolution control means and/or device according to the present invention may be of use in a broad variety of systems and/or applications, amongst them one or more of the following:
biosensors used for molecular diagnostics
rapid and sensitive detection of proteins and nucleic acids in complex biological mixtures such as e.g. blood or saliva
electrolysis to create a local pH variation for cell lysing or protein manipulation.
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

### BRIEF DESCRIPTION OF THE DRAWINGS

Additional details, characteristics and advantages of the object of the invention are disclosed in the subclaims, the figures and the following description of the respective figures and examples, which—in an exemplary fashion—show several preferred embodiments of a device according to the invention.

FIG. 1 shows a very schematic partial top view of a device according to a first embodiment of the present invention;

FIG. 2 shows a very schematic partial top view of a device according to a second embodiment of the present invention;

FIG. 3 shows a very schematic cross sectional partial view of the device of FIG. 2 somewhat along line II-II in FIG. 2 prior to the evolution of gas;

FIG. 4 shows a very schematic cross sectional partial view of the device of FIG. 3 after evolution of a certain amount of gas;

FIG. 5 shows a very schematic cross sectional partial view of the device of FIG. 3 after evolution of an amount of gas high enough to remove unbound target molecules from the capture site;

FIG. 6 shows schematically an active control means including a feedback mechanism according to one embodiment of the present invention;

FIG. 7 shows schematically an activating means according to one embodiment of the present invention; and

FIG. 8 shows a schematical perspective view of a device comprising a gas guiding means according to a further embodiment of the present invention.

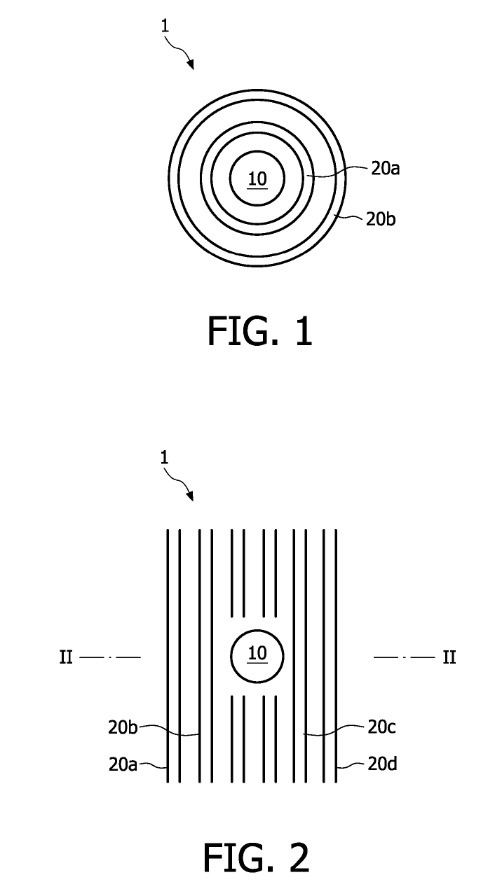

FIG. 1 shows a very schematic partial top view of a device 1 according to a first embodiment of the present invention. The device comprises a capture site 10 which is circumferentially surrounded by two electrodes 20a and 20b. It should be noted that the capture site 10 may indeed comprise a plurality of capture sites for several target molecules to be analyzed in the sample; however, the device may as well comprise several sets of capture sites which are all surrounded by electrodes. The number of electrodes per capture site is not restricted to two.

FIG. 2 shows a very schematic partial top view of a device 1 according to a second embodiment of the present invention. In this embodiment, several elongated electrodes 20a, 20b, 20c and 20d are depicted. Only a few electrodes and only one capture site is shown; however, it is clear to any skilled person in the art that most applications within the present invention will use a plurality of electrodes as well as capture sites.

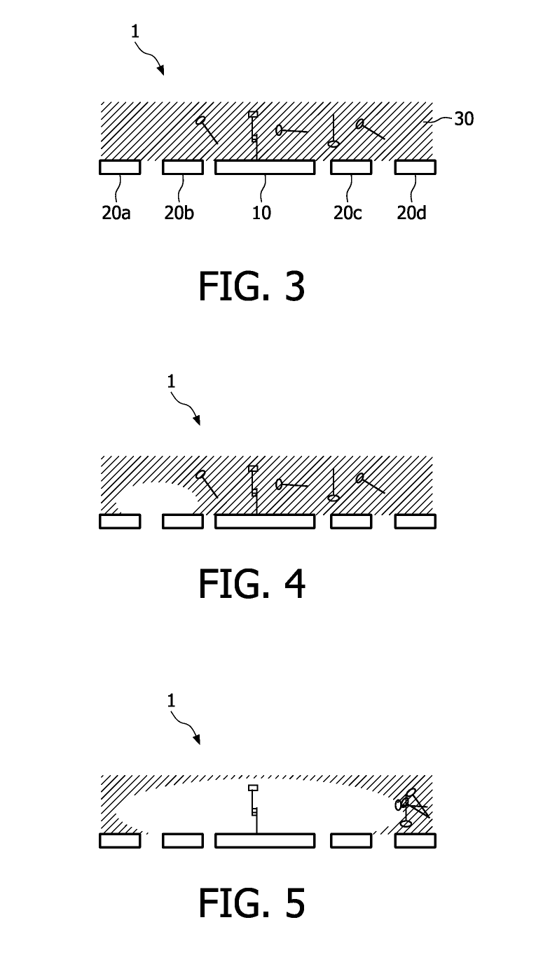

FIG. 3 shows a very schematic cross sectional partial view of the device of FIG. 2 somewhat along line II-II in FIG. 2 prior to the evolution of gas. In FIG. 3 is (very schematically) shown a target molecule which is bound to the capture site 10 (e.g. via hydrogen bonds such as in nucleic acids). The target molecule is marked with a fluorescent dye (which is “indicated” by the square). However, several other nonbound target molecules are present in the sample solution 30 which might lead to misreading of the analysis when not washed away.

FIG. 4 shows a very schematic cross sectional partial view of the device of FIG. 3 after evolution of a certain amount of gas. FIG. 5 shows a very schematic cross sectional partial view of the device of FIG. 3 after evolution of an amount of gas high enough to remove unbound target molecules from the capture site.

From FIGS. 4 and 5 it can be clearly seen that due to the evolution of gas via the electrodes, a gas bubble is created which removes the unbound target molecules whereas the bound target molecule will stay on the capture site 10 due to its binding.

The electrodes 20a-d can also be used to monitor the evolution of the gas bubble, since the gas (which is in this embodiment mainly hydrogen) has a greatly different resistance than the sample solution (which is in this embodiment essentially water). Therefore by monitoring the capacity and/or the resistance between the electrodes 20a to 20d, it can be seen how the gas bubble expands and therefore the gas “front” progresses towards the electrode 20d. When this electrode is reached, the gas evolution can be stopped since the unbound target molecules are far enough from the capture site 10.

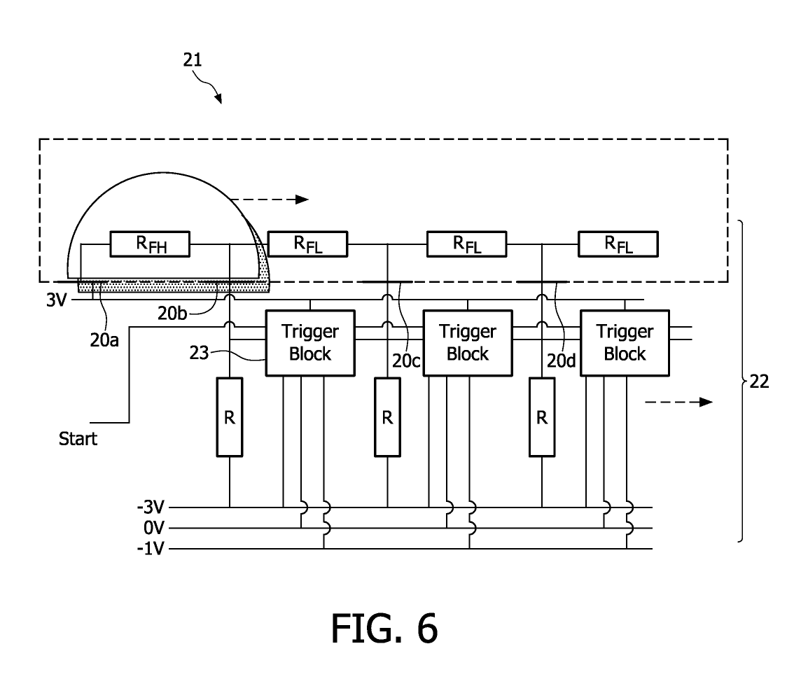

FIG. 6 shows schematically an active control circuit 21 including a feedback mechanism 22 according to an embodiment of the present invention.

In this embodiment four electrodes 20a-20d are arranged symmetrically along the capture side 10 of the device 1. Each electrode 20a-20d is connected to a resistor which serves as a reference value. An activating means 23a is assigned to compare the value of this resistant R with a value measured at the two neighboring electrodes 20a, 20b one of which is connected to a resistor R.

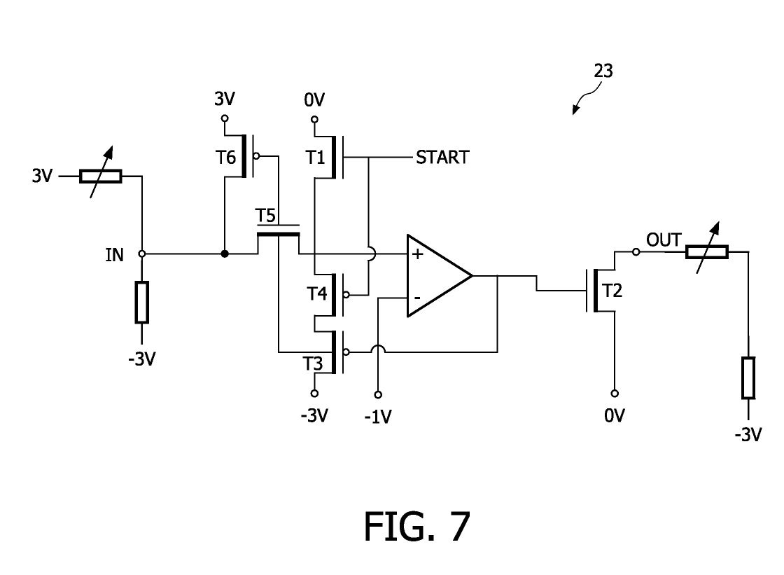

Initially there is no power supply to the circuit 21 so that no electrolysis occurs in the vicinity of the electrodes 20a-20d. As the power supply in this embodiment is exemplarily moved to 3V the start line is pulsed. In this state the voltage between electrodes 20a and 20b is 3V and the potential between the other following electrodes 20c to 20n is 0V. As the start pulse goes low the voltage of the fluid resistance R_{FL} and the resistor R determines the voltage at electrode 20b. These two values are compared in the activating means 23, in this embodiment a trigger block (e.g. FIG. 7). As long as the electrode is surrounded by the fluid of the sample there will be no great differences between these two values. As soon as the bubble front of the gas bubble evolved at the electrode 20a reaches and surrounds electrode 20b the resistance between these two electrodes increases, i.e. it changes from R_{FL} to R_{FH} the trigger block 23 measures a drop of the voltage at electrode 20b. This difference causes the trigger block 23 to fire. It then forces the electrode 20b to 3V and enables the potential divider action between the fluid resistance R_{FL} of the electrode 20b and 20c and the resistor R attached to electrode 20c, These steps repeat until the end of the electrode structure. If desired the process can be stopped anytime by initiating a high voltage at a following electrode (correct if it is not possible or if there are more possibilities to stop).

Once the resistance R_{FL} goes high the electrode 20a can be automatically brought to a high voltage to stop electrolysis between two electrodes 20a and 20b and to cause electrolysis between the next two electrodes 20b and 20c and thereby enlarge the gas bubble in the desired direction. After a short period of time after the electrode 20a was shut down the gas will be absorbed by the fluid.

In some embodiments it may be preferred not to stop the gas evolution by shutting down the electrodes after the gas “front” has reached the next electrode, for example if the gas should be sustained to avoid a reduction of the volume occupied by the gas. This can be for example advantageous in a chamber in which the gas is utilized to move a piston forward and to hold it in this position or move it further. If on the other hand the gas is not sustained in the chamber a negative pressure can emerge and possibly move the piston oppositely.

The starting point of electrolysis may be different from time to time. Therefore in an alternative embodiment of the present invention the power supply is not moved directly to 3V but first to a lower voltage for example 1V. If electrolysis does not occur at this voltage the start pulse will just be repeated with a slightly higher voltage. This will be iteratively repeated until electrolysis and therefore a gas formation is achieved. The voltage at which the gas formation starts may be higher or lower depending on several circumstances as the surface of the electrodes for example a significant roughness, whether a double layer of charge forms on the electrode, conductivity of the fluid, or possibly whether a partially insulating layer forms on the electrode with an associated voltage drop.

FIG. 7 shows exemplarily a possible design of a trigger block 23 according to FIG. 6. For a person skilled in the art it will be obvious that the details of this trigger block 23 can be substituted with a plurality of alternatives known in the art.

In this special embodiment the start pulse is applied to all of the trigger blocks 23 and its action is to pull the input of a comparator 24 to 0V. This causes the output of the comparator 24 to go high which connects tan IN terminal 25 to the comparator 24 (which is also initially at 0V) and the Output 26 to 0V. When the start pulse goes low, the input voltage IN is the voltage caused by the potential divider of the fluid resistance R_{FL} and the resistance R. As it drops as the gas bubble evolves it reaches the switch point of the comparator for example by −1V and causes the output OUT of the comparator to go low. This turns off the connection of OUT to 0V so the next stage trigger can function and it also pulls IN high to 3V to stop electrolysis at the input. The input to the comparator is also pulled to −3V to ensure the comparator output remains low.

The comparator can easily be constructed using standard techniques. A technique used often in LTPS circuitry is to use an inverter and capacitor in switched mode to enable the −1V reference voltage to be stored as the switch point of the inverter.

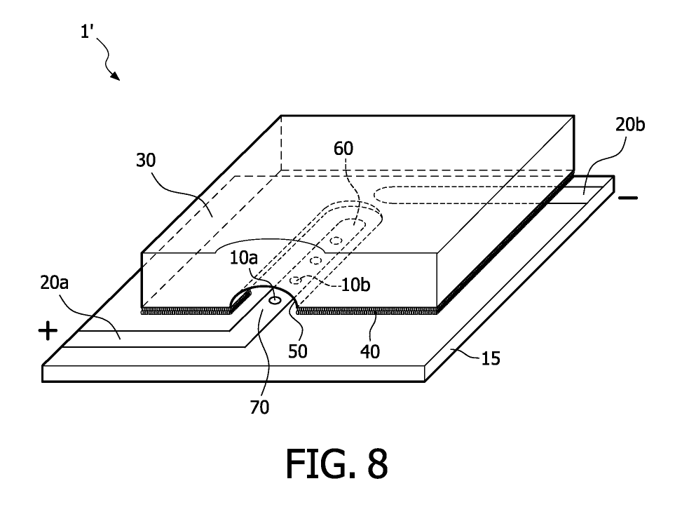

FIG. 8 shows a schematical perspective view of a device 1′ comprising a gas guiding means according to a further embodiment of the present invention. The device comprises a glass substrate 15, on which two electrodes 20a, 20b are provided. It should be noted that the device 1′ is highly schematical and in most actual applications the number of electrodes will most likely be much higher.

The gas guiding means comprises a hydrogel material 30, which is linked to the glass substrate 15 via a connecting material 40. The connecting material 40 is preferably as described above. The part of the glass substrate which is covered with the electrodes 20a, 20b is essentially non-linked to the hydrogel material, so that—if gas is produced—the hydrogel material will form a channel 50. It should be noted that the channel 50 is highly schematical as well and in most application the shape and dimension of the channel will most likely be different.

According to a -non limiting-example, the hydrogel material was produced out of a mixture of 80% demineralized water and 20% acryl amide/N,N-methylene bisacrylamide (in a 5:1 ratio) was mixed and 2 wt % of Irgacure 2959 (photoinitiator) was added.

The connecting material 40 was trimethoxysilyl-propyl-methacrylate.

On the device, several capture sites (two of which are referred to as 10a and 10b) are present. The device layout is set that way that the capture sites are provided on one electrode 20a, where hydrogen will be released when current is applied. On the other electrode 20b, where oxygen will be released, no capture sites are present.

It should be noted that the hydrogel material is set up that way that oxygen is allowed to dissorb through the hydrogel material whereas hydrogen cannot wander through the hydrogel material.

Upon applying current, hydrogen be started to be produced around part 60 of the electrode. The hydrogel material is in this embodiment set up that way that only gas bubbles which have a certain size are wandering away from the end part 60 of the electrode.

When gas bubbles (not shown in the Figs.) are produced in an appropriate size and number, they will wander from the end part 60 of the electrode 20a through the channel 50 until the part 70 of the electrode, where the hydrogen is set free. Since in the path of the hydrogen the capture sites 10a, 10b are present, the unbound target molecules will be removed; due to the layout of the device 1′ all unbound target molecules will therefore be removed from all capture sites 10a, 10b in “a single step”.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. A device for analyzing one or more samples, especially fluid samples for the presence, amount or identity of one or more target molecules in the samples, comprising one or more capture sites whereby the device comprises a gas evolving means for washing away and/or removing unbound target molecules and/or redundant sample fluid from the capture site(s).

2. The device according to claim 1, whereby the gas evolved by the gas evolving means is created by electrolysis of the sample solution.

3. The device according to claim 1, whereby the gas evolving means evolves gas by applying a voltage of ≧1.2 V and ≦10V.

4. The device according to claim 1, whereby the gas evolving means is located in a distance of ≧0 mm and ≦5 mm from the capture sites.

5. The device according to claim 1, whereby the gas evolving means comprises at least one pair of electrodes.

6. Gas evolution control means in particular for removing unbound target molecules and/or redundant sample fluid from at least one capture site of a device according to claim 1 including at least one feedback mechanism controlling the formation and/or the movement of evolved gas bubbles.

7. Gas evolution control means according to claim 6 comprising at least two electrodes for said gas evolution, wherein the feedback mechanism comprises an impedance network of at least one capacitor and/or resistor which is/are located between said electrodes to measure a capacity and/or a resistance between said electrodes to control the formation and/or the movement of the gas bubble.

8. The device according to claim 1, furthermore comprising a gas guiding means.

9. The device according to claim 1, whereby the gas guiding means comprises a hydrogel and/or nanoporous material.

10. A system incorporating a device according to claim 1 and being used in one or more of the following applications:
biosensors used for molecular diagnostics
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
